(19) United States
(12) Patent Application Publication
(10) Pub. No.: US 2025/0295909 A1
(43) Pub. Date: Sep. 25, 2025

(54) APPARATUS AND METHODS TO TREAT MENOPAUSE AND ASSOCIATED SYMPTOMS BY MODULATING NERVE ACTIVITY

(71) Applicant: Vasu Sharma, Hillsboro, OR (US)

(72) Inventor: Vasu Sharma, Hillsboro, OR (US)

(21) Appl. No.: 19/084,781

(22) Filed: Mar. 20, 2025

Related U.S. Application Data
(60) Provisional application No. 63/568,245, filed on Mar. 21, 2024.

(51) Int. Cl. A61N 1/04; A61N 1/36

(52) U.S. Cl. CPC A61N 1/0456; A61N 1/36034

(57) ABSTRACT

Methods for modulating nerve activity to treat menopause and associated symptoms are disclosed. The methods include placing a neuromodulation device in communication with a target site of the autonomic nervous system or parasympathetic nervous system. The neuromodulation device comprises at least a modulating component, at least one controller, and at least one connecting element. The device delivers a stimulation paradigm to the target site, modulating nerve activity to induce a therapeutic effect, thereby treating menopause and associated symptoms.

### CROSS-REFERENCE TO RELATED APPLICATIONS

This application claims the benefit of co-pending U.S. Provisional Patent Application No. 63/568,245, which was filed on Mar. 21, 2024. The entire content of the foregoing provisional patent application is incorporated herein by reference.

### TECHNICAL FIELD

The present disclosure relates to an apparatus and methods to modulate nerve activity and, in particular, to invasive and non-invasive apparatus, methods and systems for modulating the nerve activity to treat menopause and associated symptoms.

### BACKGROUND

Menopause is a natural biological phenomenon marking the end of a woman's menstrual cycles, typically occurring in her late 40s or early 50s. This physiological transition gives rise to symptoms like hot flashes, mood fluctuations, changes in bone density etc. Hot flashes stand out as a predominant symptom significantly impacting women's daily lives. Hot flashes are intense waves of heat and can lead to severe discomfort, disrupted sleep and increased irritability, making them a primary focus for interventions aimed at improving the quality of life of menopausal women. Furthermore, hot flashes can be triggered by various other factors/conditions, including but not limited to hormonal changes during pregnancy, thyroid disorders, medications, emotional stress and notably, androgen deprivation therapy (ADT) in prostate cancer treatment.

Hormone replacement therapy (HRT) has been a primary approach for managing menopausal symptoms by supplementing declining estrogen levels. While effective in alleviating symptoms and enhancing quality of life, HRT comes with significant drawbacks. HRT use has been associated with potential health risks, including an increased likelihood of blood clots, strokes and breast cancer. The ongoing debate surrounding the safety of HRT has opened an opportunity for exploring innovative solutions. Today, healthcare professionals and patients are considering technology and medical device solutions as promising alternatives, offering a safer approach for navigating the challenges of menopause. Notably, the desire for non-pharmaceutical products reflects a growing preference among patients for solutions with fewer associated complications.

Bioelectronic medicine is an emerging therapeutic modality where electrical interventions replace traditional chemical interventions or drugs or medications. Using one or more electrodes that either penetrate a neural target or are in close proximity to it, nerve activity can be modulated to elicit a therapeutic effect. Market insights indicate that menopausal women prefer device-based, non-chemical solutions for managing symptoms. However, no chemical-free, device-based therapy with proven therapeutic effects currently exists on the market. Therefore, bioelectronic medicine, also known as neuromodulation therapy, presents a promising therapeutic modality for menopausal symptoms.

### SUMMARY

In accordance with embodiments of the present disclosure, an exemplary apparatus for treating menopause and associated symptoms by modulating nerve activity is provided. The apparatus generally includes a neuromodulation device that may be used to deliver a stimulation paradigm to a target site of autonomic nervous system (ANS) or parasympathetic nervous system (PNS), where the target site is directly or indirectly associated with menopause and/or its associated symptoms. The apparatus may comprise of at least a modulating component (comprising one or more energy delivery elements), at least one controller (or programmer), and at least one connecting element (e.g., wires). The modulating component can either stimulate or inhibit the nerve or neural target and the connecting element can help in connecting the modulating component to the controller (or programmer). Each component of the apparatus can be in communication with one another using both physical connecting elements or non-physical connecting elements. In some embodiments, the apparatus may use a non-invasive (transcutaneous), minimally invasive and/or invasive approach.

An energy delivery element, e.g., electrode, transducer, coil, light source, ultrasound emitter, drug delivery system, thermal module, mechanical actuator, etc., can be controllable to generate stimulation paradigms with parameters that may be varied, wherein such parameters are unique to each energy modality. For electrical stimulation, adjustable parameters may include frequency, voltage, current, pulse width, intensity, duty cycle, waveform shape, polarity, burst characteristics, etc. Similarly, other energy modalities have parameters unique to the modality. In some embodiments, the apparatus can be configured to deliver magnetic energy, chemical energy, electrical energy, ultrasound energy, optical energy, thermal energy, mechanical energy, radiofrequency energy, or any other known or future-discovered form of energy, either individually or in any functional combination thereof.

In some embodiments, the apparatus and/or its components may be designed to have dimensions and/or size suitable for application to a particular target site of the ANS or PNS. For example, the electrode can have dimensions and/or size suitable for the vagus nerve (cranial nerve X), its branches (e.g., auricular, pharyngeal, laryngeal, cardiac, pulmonary, esophageal, and abdominal branches), the dorsal motor nucleus of the vagus, the nucleus ambiguus, intramural ganglia, and/or any combinations thereof.

In some embodiments, one or more controllers may deliver a stimulation paradigm to the energy delivery elements that may be constant, varying, and/or modulated with respect to parameters including, but not limited to, current, voltage, frequency, pulse width, waveform, cycle, amplitude, etc. In some embodiments, the current may range from approximately 0.001 to 100,000 microamperes (μA). In some embodiments, the voltage may range from approximately 0.01 millivolt (mV) to 50 volts (V). In some embodiments, the frequency for electrical stimulation may range from approximately 0.01 Hz to 100 kHz. In some embodiments, the pulse-width may range from approximately 1 to 10,000 microseconds (μs).

In some embodiments, the energy delivery elements or electrodes may be configured as monopolar, bipolar or multipolar. In some embodiments, an apparatus may comprise one or more multipolar energy delivery elements or electrodes with multiple exposed contacts to enhance therapeutic efficacy and more precise targeting of the indication.

Anecdotal evidence in a peer-reviewed study suggests that electrical stimulation with frequency below 10 Hz might induce hot flashes. A highly skilled person in the art would recognize that this means electrical stimulation frequencies above 10 Hz may be used to counteract or mitigate hot flashes.

In accordance with aspects of the present disclosure, an exemplary method for treating menopause and associated symptoms by modulating nerve activity is provided. The method generally includes a framework (or approach, modality, or process) that may be employed to practice the present disclosure. In some aspects, the method may involve modulating the activity of a nerve or neural target at one or more sites within the autonomic nervous system (ANS) or parasympathetic nervous system (PNS), where such nerve or neural target is directly or indirectly associated with menopause and/or its associated symptoms. In some embodiments, the method can be practiced via a non-invasive (transcutaneous), minimally invasive, transdermal, percutaneous, intravascular and/or invasive approach.

In some aspects, it may be desirable to modulate nerve activity by placing an apparatus into electrical communication with only one specific parasympathetic nervous tissue or nerve structure, for e.g., auricular branch of the vagus nerve (ABVN) located in cymba concha of the ear. This can influence central autonomic regulation, nerve excitability, neurotransmitter levels, synaptic plasticity, neuroplasticity, and neuronal activity at target tissues. These target tissues may include, but are not limited to, the hypothalamus and median preoptic nucleus (MnPO), which are key regions involved in thermoregulation, neuroendocrine control, and autonomic homeostasis.

In some aspects, delivery of a stimulation paradigm to a target site of the ANS or PNS, e.g., vagus nerve, can increase vagal excitability, thereby facilitating reliable neural signaling, enhanced information transfer, and activity-dependent neuroplasticity in vagal projection sites. This modulation may result in adaptive changes in synaptic strength, activity-dependent plasticity, and circuit-level reorganization, leading to sustained improvements in neuroendocrine function, thermoregulation, cardiovascular control, inflammatory responses, cognitive function, mood regulation, metabolic processes, and other physiological functions associated with menopause and its associated symptoms.

Prior applications of stimulating the vagus nerve have primarily focused on treating neurological and autonomic conditions such as epilepsy, depression, and inflammation-related disorders. However, vagus nerve stimulation (VNS) has not been previously explored as a therapy for menopause and its associated symptoms. Preclinical evidence suggests that estrogen deficiency (or fluctuation) results in reduced excitability of vagal afferents, an effect that has not been previously investigated in the context of treating menopause, hot flashes, and/or other symptoms of menopause. Unlike prior VNS applications that focus on broad autonomic or inflammatory modulation, the present disclosure establishes a novel use of VNS for addressing menopause and its associated symptoms. By leveraging VNS to compensate for menopause-associated vagal hyporesponsiveness, this disclosure provides a new therapeutic approach distinct from existing pharmacological and non-pharmacological interventions.

Other features, embodiments and methods will become apparent from the following detailed description considered in conjunction with the accompanying drawings. It is to be understood, however, that the drawings are designed as an illustration only and not as a definition of the limits of this disclosure.

### BRIEF DESCRIPTION OF THE DRAWINGS

To assist those of skill in the art in making, using and understanding the disclosed apparatus and methods, reference is made to the accompanying figures, wherein:

FIG. 1 is a schematic illustration showing the vagus nerve (cranial nerve X) and its major branches, including the auricular, pharyngeal, laryngeal, cardiac, esophageal, pulmonary, and abdominal branches.

FIG. 2 is a process flow diagram illustrating a method for treating menopause, its symptoms, and/or related conditions in a subject by modulating nerve activity, according to one aspect of the present disclosure.

FIG. 3 is a process flow diagram illustrating a method for treating menopause, its symptoms, and/or related conditions in a subject by modulating nerve activity, according to one aspect of the present disclosure.

FIG. 4 is a process flow diagram illustrating a method for treating menopause, its symptoms, and/or related conditions in a subject by modulating nerve activity, according to one aspect of the present disclosure.

FIG. 5 is a perspective view of several exemplary energy delivery elements for treating menopause, its symptoms, and/or related conditions in a subject by modulating nerve activity, in accordance with the present disclosure.

FIG. 6 is a process flow diagram illustrating a method for treating menopause, its symptoms, and/or related conditions in a subject by modulating nerve activity, according to one aspect of the present disclosure.

FIG. 7 is a process flow diagram illustrating a method for treating menopause, its symptoms, and/or related conditions in a subject by modulating nerve activity, according to one aspect of the present disclosure.

FIG. 8 is a process flow diagram illustrating a method for treating menopause, its symptoms, and/or related conditions in a subject by modulating nerve activity, according to one aspect of the present disclosure.

FIG. 9 is a process flow diagram illustrating a method for treating menopause, its symptoms, and/or related conditions in a subject by modulating nerve activity, according to one aspect of the present disclosure.

FIG. 10 is a process flow diagram illustrating a method for treating menopause, its symptoms, and/or related conditions in a subject by modulating nerve activity, according to one aspect of the present disclosure.

### DETAILED DESCRIPTION

### Key Definitions

Unless otherwise stated, all scientific and technical terms used herein have the same meaning as commonly understood by a person skilled in the art to which this disclosure pertains.

In the context of the present disclosure, the terms ‘autonomic nervous tissue’, ‘autonomic nervous system’, ‘ANS’, ‘sites within autonomic nervous system’, ‘sites within ANS’, ‘neural target at sites within autonomic nervous system’, ‘neural target at sites within ANS’, ‘nerve at sites within autonomic nervous system’, ‘nerve at sites within ANS’, as well as other similarly worded terms or phrases can refer to any tissues of the autonomic nervous system including, but not limited to, neurons, axons, fibers, tracts, nerves, plexus, afferent plexus fibers, efferent plexus fibers, ganglia, pre-ganglionic fibers, post-ganglionic fibers, the vagus nerve (cranial nerve X), its branches (e.g., auricular, pharyngeal, laryngeal, cardiac, pulmonary, esophageal, and abdominal branches), the dorsal motor nucleus of the vagus, the nucleus ambiguus, intramural ganglia, and/or any combinations thereof.

In the context of the present disclosure, the terms ‘parasympathetic nervous tissue’, ‘parasympathetic nervous system’, ‘PNS’, ‘sites within parasympathetic nervous system’, ‘sites within PNS’, ‘neural target at sites within parasympathetic nervous system’, ‘neural target at sites within PNS’, ‘nerve at sites within parasympathetic nervous system’, ‘nerve at sites within PNS’, as well as other similarly worded terms or phrases can refer to any tissues of the parasympathetic nervous system including, but not limited to, neurons, axons, fibers, tracts, nerves, plexus, afferent plexus fibers, efferent plexus fibers, ganglia, pre-ganglionic fibers, post-ganglionic fibers, the vagus nerve (cranial nerve X), its branches (e.g., auricular, pharyngeal, laryngeal, cardiac, pulmonary, esophageal, and abdominal branches), the dorsal motor nucleus of the vagus, the nucleus ambiguus, intramural ganglia, and/or any combinations thereof.

In the context of the present disclosure, the terms ‘vagus nerve’, ‘cranial nerve X’, ‘sites within vagus nerve’, ‘neural target at sites within vagus nerve’, ‘nerve at sites within vagus nerve’, as well as other similarly worded terms or phrases can refer to any tissues of the vagus nerve including, but not limited to, neurons, axons, fibers, tracts, nerves, plexus, afferent plexus fibers, efferent plexus fibers, ganglia, pre-ganglionic fibers, post-ganglionic fibers, the vagus nerve (cranial nerve X), its branches (e.g., auricular, pharyngeal, laryngeal, cardiac, pulmonary, esophageal, and abdominal branches), the dorsal motor nucleus of the vagus, the nucleus ambiguus, intramural ganglia, and/or any combinations thereof.

As used herein, the term ‘subject’ can be used interchangeably with the term ‘patient’ and refer to any warm-blooded organism including, but not limited to, human beings, pigs, rats, mice, dogs, goats, sheep, horses, monkeys, apes, farm animals, livestock, rabbits, cattle, etc.

As used herein, the terms ‘modulate’, ‘modulating’, ‘modulated’, ‘modulation’, ‘modulator’, ‘neuromodulation’, ‘neuromodulatory effects’ with reference to an autonomic nervous tissue or parasympathetic nervous tissue can refer to causing a change in neuronal activity, chemistry and/or metabolism. The change can refer to an increase, decrease, or even a change in a pattern of neuronal activity. The terms may refer to either excitatory or inhibitory stimulation, or a combination thereof, and may be at least electrical, magnetic, ultrasound, optical, chemical, thermal, mechanical, radiofrequency, or any other known or future-discovered form of energy or a combination of two or more of these. These terms can also be used to refer to a masking, altering, overriding, or restoring of neuronal activity. Other terms or phrases that have similar wording as the terms described in this paragraph may also convey the description given herein.

As used herein, the term ‘neuromodulation device’ refers to any apparatus, system, or component designed to deliver energy-based stimulation paradigm to a nerve or neural target to modulate its activity. A neuromodulation device may be invasive, minimally invasive, or non-invasive and may utilize one or more energy modalities, including but not limited to electrical, magnetic, ultrasound, optical, chemical, thermal, mechanical, or radiofrequency stimulation, or any other known or future-discovered form of energy. Other terms or phrases that have similar wording as the term described in this paragraph may also convey the description given herein.

As used herein, the terms ‘in communication’, ‘establish communication’, ‘establishing communication’, ‘establishes communication’, ‘electrical communication’, as well as other similarly worded terms or phrases can refer to the ability of an energy field generated by one or more energy delivery elements (e.g., electrode, electrode array, transducer, coil, light source, ultrasound emitter, drug delivery system, thermal module, mechanical actuator) to be transferred, or to have a neuromodulatory effect, within and/or on autonomic or parasympathetic nervous tissue (e.g., vagus nerve). These terms can also refer to at least a portion of an apparatus being adjacent, in the general vicinity, in close proximity, or directly next to and/or directly on or in a target site of autonomic nervous tissue or parasympathetic nervous tissue. In some embodiments and/or aspects, the term can mean that at least a portion of an apparatus is ‘in communication’ with a target site of the autonomic nervous system or parasympathetic nervous system if application of a stimulation paradigm (e.g., an electrical, magnetic, ultrasound, optical, chemical, thermal, mechanical, radiofrequency, or a combination of two or more of these) thereto results in a modulation of neuronal activity to elicit a desired response, such as treating a symptom associated with menopause.

As used herein, the terms ‘menopause’, ‘menopause and associated symptoms’, menopause and/or associated symptoms’, ‘menopause and its associated symptoms’, ‘menopause and/or its associated symptoms’, ‘menopause, its symptoms, and/or related conditions’, ‘menopause, or at least a symptom associated therewith’, ‘menopause, or at least the symptoms that characterize the menopause’, ‘symptom of menopause’, ‘effects of menopause’, ‘menopause and/or one or more associated symptoms and/or one or more related or unrelated medical indications’, ‘menopause or one or more of its associated symptoms’, ‘one or more symptoms associated with the menopause’, ‘one or more symptoms of menopause’, ‘menopause, its associated symptoms (e.g., hot flashes), and/or related conditions’, ‘symptom associated with menopause’, as well as other similarly worded terms or phrases can refer to any disease/s, disorder/s, sign/s, and/or symptom/s that is/are related to, caused at least in part by, and/or correlated with menopause. Menopause is generally defined as the last natural menstrual period, and is characterized by the cessation of ovarian function, leading to the substantial diminution of circulating estrogen in the bloodstream. Menopause can also be associated with conditions of decreased estrogen production that may be caused by surgical means, chemical means, and/or a disease state that leads to premature diminution or cessation of ovarian function. Non-limiting examples of the list of above mentioned terms can include cardiovascular disease (e.g., hypertension, high cholesterol), vasomotor symptoms including hot flashes, vaginal or Vulvar atrophy, myalgia, arthralgia, atrophic vaginitis, vaginal dryness, pruritus, dyspareunia, dysuria, insomnia, irritability, frequent urination, urinary incontinence, urinary tract infections, dysfunctional uterine bleeding, infertility, osteoarthritis, bone weakness, brittleness or osteoporosis, memory loss, depression, etc., and other disease processes that many women begin to experience or begin to develop during the menopausal period.

As used herein, the terms ‘indication’ or ‘indications’ refer to a medical condition, disease, disorder, syndrome, symptom, or physiological state for which a treatment, therapy, intervention, or neuromodulation technique is intended, proposed, or applied. An indication may include, but is not limited to, primary medical conditions, secondary complications, risk factors, or any associated symptoms that can be treated by neuromodulation or other therapeutic interventions. Other terms or phrases that have similar wording as the term described in this paragraph may also convey the description given herein.

As used herein, the terms ‘energy delivery element’ or ‘energy delivery elements’ refer to one or more components of a neuromodulation device that are configured to deliver energy to a nerve or neural target non-invasively (e.g., transcutaneously), minimally invasively, or invasively. In invasive or minimally invasive applications, energy delivery elements may be intraneural (e.g., penetrating nerve) and/or extraneural (e.g., not penetrating nerve). Energy delivery elements may include, but are not limited to, electrodes, coils, transducers, emitters, optical fibers, ultrasound arrays, chemical reservoirs, thermal applicators, mechanical actuators, or any other structures designed to transmit electrical, magnetic, electromagnetic, acoustic, optical, chemical, thermal, mechanical, radiofrequency, or other forms of energy to a target site. Other terms or phrases that have similar wording as the term described in this paragraph may also convey the description given herein.

As used herein, the term ‘modulating component’ refers to one or more structures, assemblies, or subsystems that comprise at least one energy delivery element and are configured to deliver a stimulation paradigm to a neural target. A modulating component is responsible for delivering energy in a controlled manner to achieve neuromodulation and may be designed for non-invasive (e.g., transcutaneous), minimally invasive, and/or invasive applications. Other terms or phrases that have similar wording as the term described in this paragraph may also convey the description given herein.

As used herein, the terms ‘apparatus’ or ‘apparatuses’ refer to one or more devices, systems, or combinations of components designed to perform a specific function, such as modulating the nerve (or neural target) activity. Other terms or phrases that have similar wording as the terms described in this paragraph may also convey the description given herein.

As used herein, the term ‘source of energy’ refers to any system, component, or reservoir capable of storing and/or generating energy. It may either store energy for use or generate energy in real-time. A source of energy may be internal or external and may function independently or as part of an integrated system. Other terms or phrases that have similar wording as the term described in this paragraph may also convey the description given herein.

As used herein, the terms ‘connecting element’ or ‘connecting elements’ refer to one or more structures, components, or interfaces that facilitate the transfer of energy, signals, and/or information between a source of energy and an energy delivery element, or between different components (e.g., a controller) of a neuromodulation device. A connecting element may establish a physical connection (e.g., wired) or a non-physical connection (e.g., wireless). Other terms or phrases that have similar wording as the terms described in this paragraph may also convey the description given herein.

As used herein, the term ‘cymba concha’ refers to the upper, deeper region of the external ear's concha, located superior to the cavum concha and adjacent to the crus of the helix. The cymba concha is a site of anatomical and functional significance due to its rich innervation by the auricular branch of the vagus nerve (ABVN), making it a viable target for non-invasive (e.g., transcutaneous) and minimally invasive vagus nerve stimulation techniques. Other terms or phrases that have similar wording as the term described in this paragraph may also convey the description given herein.

As used herein, the terms ‘hot flash’, ‘hot flashes’, ‘hot flush’, ‘hot flushes’ and ‘night sweats’ can be used interchangeably to refer to events that impact blood vessel diameter and are characterized by the sudden onset of intense warmth that typically begins in the chest and progresses to the neck and face. Hot flashes are often accompanied with palpitations, profuse sweating, and red blotching of the skin. A hot flash can begin when regions of the brain that regulate body temperature begin to react to various issues, such as a drop in hormone level and effect of chemotherapy. The median preoptic nucleus (MnPO) of the hypothalamus, which plays a critical role in body's thermoregulatory function, becomes dysfunctional. As per one of the explanations, as a result, the temperature signals/information from different regions of the body fail to reach the brain reliably i.e., the signal strength reduces. Therefore, body/brain can misinterpret the incoming temperature signal/s, mistakenly perceiving an urgent need for cooling. In response, the autonomic nervous system (ANS) triggers/initiates a series of downstream effects, including but not limited to dilation of blood vessels (vasodilation) and increased blood flow, which can result in dizziness and anxiety. The affected individual feels intensely hot, primarily in the upper portions of his or her body. The Sweat glands activate and blood rushes to the extremities and face, neck and chest. The peripheral blood vessels dilate and causes sweating.

As used herein, the terms ‘treat’ or ‘treating’ can refer to therapeutically regulating, preventing, improving, alleviating the symptoms of, and/or reducing the effects of menopause in a subject, such as a perimenopausal, menopausal, or postmenopausal subject. As such, treatment can also include situations where menopause, or at least a symptom associated therewith, is completely inhibited, e.g., prevented from happening or stopped (e.g., terminated) such that the subject no longer suffers from at least one or more of the symptoms that characterize the menopause. Other terms or phrases that have similar wording as the terms described in this paragraph may also convey the description given herein.

As used herein, the terms ‘stimulation paradigm’ or ‘stimulation paradigms’ refer to a structured protocol that defines the pattern and manner in which energy is delivered to a neural target to achieve a physiological or therapeutic effect. A stimulation paradigm consists of one or more parameters that govern the characteristics of the applied energy, such as waveform, amplitude, frequency, pulse width, duty cycle, phase, timing, duration, and mode of delivery (e.g., continuous, periodic, adaptive, or closed-loop). Each energy modality (e.g., electrical, magnetic, electromagnetic, acoustic, optical) has unique parameters specific to the modality, which can be configured to achieve distinct neuromodulatory effects. The specific configuration of these parameters can influence neural excitability, neurotransmitter release, synaptic activity, and/or other physiological responses in a subject. Other terms or phrases that have similar wording as the terms described in this paragraph may also convey the description given herein.

As used herein, the singular forms ‘a’ ‘an’ and ‘the’ can also include the plural forms, unless the context makes the otherwise obvious. It will also be understood that the terms ‘comprises’ and/or ‘comprising’, as used herein, can specify the presence of stated features, steps, operations, elements, and/or components, but do not preclude the presence or addition of one or more other features, steps, operations, elements, components, and/or groups thereof.

As used herein, the term ‘and/or’ can include any and all combinations of one or more of the listed items.

As used herein, phrases such as ‘between X and Y’ and ‘between about X and Y’ can be interpreted to include Xand Y.

As used herein, phrases such as ‘between about X and Y’ can mean ‘between about X and about Y.’ As used herein, phrases such as ‘from about X to Y’ can mean ‘from about X to about Y.’

It will be understood that when an element is referred to as being ‘on’, ‘attached’ to, ‘connected’ to, ‘coupled’ with, ‘contacting,’ etc., another element, it can be directly on, attached to, connected to, coupled with or contacting the other element or intervening elements may also be present. In contrast, when an element is referred to as being, for example, ‘directly on,’ ‘directly attached’ to, ‘directly connected’ to, ‘directly coupled’ with or ‘directly contacting’ another element, there are no intervening elements present. It will also be appreciated by those of skill in the art that references to a structure or feature that is disposed ‘directly adjacent’ another feature may have portions that overlap or underlie the adjacent feature, whereas a structure or feature that is disposed ‘adjacent’ another feature may not have portions that overlap or underlie the adjacent feature.

Spatially relative terms, such as ‘under,’ ‘below,’ ‘lower,’ ‘over,’ ‘upper’ and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as ‘under’ or ‘beneath’ other elements or features would then be oriented ‘over’ the other elements or features.

It will be understood that, although the terms ‘first,’ ‘second’ etc. may be used herein to describe various elements, these elements should not be limited by these terms or sequence. These terms are only used to distinguish one element from another. Thus, a ‘first’ element discussed below could also be termed a ‘second’ element without departing from the teachings of the present disclosure. The sequence of operations (or steps) is not limited to the order presented in the claims or figures unless specifically indicated otherwise.

### Description of Exemplary Embodiments

In accordance with embodiments of the present disclosure, an exemplary apparatus for treating menopause and associated symptoms by modulating nerve activity is provided. The apparatus generally includes a neuromodulation device that may be used to practice the present disclosure. In some embodiments, an apparatus (e.g., neuromodulation device) or its components may be positioned in close proximity to a target site of the autonomic nervous system (ANS) or parasympathetic nervous system (PNS) that is directly or indirectly associated with menopause and/or its associated symptoms. The term “target site of the autonomic nervous system (ANS) or parasympathetic nervous system (PNS) that is directly or indirectly associated with menopause and/or its associated symptoms” refers to any site within, or in communication with, the ANS or PNS where modulating the nerve activity is produces a therapeutic outcome for menopause and associated symptoms.

In some embodiments, an apparatus that may be used to practice the present disclosure can be external (non-invasive or minimally invasive) and configured to be placed on or adjacent to the skin of a subject, in close proximity of a target site of the ANS or PNS that is directly or indirectly associated with menopause and/or its associated symptoms. In some embodiments, an apparatus can be temporarily or permanently implanted, within, on, or otherwise associated with a subject experiencing or suspected of experiencing menopause and associated symptoms.

The apparatus of the present disclosure can be configured to deliver various types of stimulation paradigms to neural targets within, or in communication with ANS or PNS that is directly or indirectly associated with menopause and/or its associated symptoms. In some embodiments, the apparatus of the present disclosure can be configured to deliver only magnetic energy, only electrical energy, only chemical energy, only ultrasound energy, only optical energy, only thermal energy, only mechanical energy, only radiofrequency energy, or any other known or future-discovered form of energy, either individually or in any functional combination thereof. In some embodiments, the apparatus of the present disclosure can comprise at least one energy delivery element (e.g., electrode, transducer, coil, light source, ultrasound emitter, drug delivery system, thermal module, mechanical actuator) and a source of energy, which is in communication with at least one energy delivery element and can produce one or more stimulation paradigms. In some embodiments, an apparatus can include a storage chamber containing a pharmacological or biological or chemical agent, an appropriate delivery mechanism and apparatus. Pharmacological or biological or chemical agents include, but are not limited to, compounds, small or large molecule drugs, biologics, nucleic acids (e.g., DNA, RNA, mRNA, siRNA, CRISPR-associated molecules), peptides, polypeptides, proteins, antibodies, enzymes, lipids, carbohydrates, hormones, growth factors, stem cells (or other cell-based therapies), exosomes, synthetic or bioengineered cells, nanoparticles, toxins, viral or non-viral vectors. In some embodiments, an apparatus can use various other energy-based modalities, including but not limited to ultrasound, radiofrequency, electromagnetic waves, electric fields, magnetic fields, photobiomodulation, infrared, visible light, laser energy, optogenetic stimulation, cryotherapy, thermal energy, mechanical stimulation, piezoelectric stimulation, acoustic waves, plasma energy, ionizing and non-ionizing radiation, and any other known or future-discovered therapeutic modality. It should be understood that the therapeutic modalities described herein may be utilized either individually or in any functional combination thereof within the scope of the present disclosure.

In some embodiments, an apparatus can comprise of a modulating component (comprising one or more energy delivery elements), at least one controller (or programmer), and at least one connecting element. The modulating component can either stimulate or inhibit the nerve or neural target and the connecting element (e.g., wires) can help in connecting the modulating component to the controller (or programmer). In some embodiments, each component of the apparatus can be in communication with one another using both physical connecting elements (e.g., wires) and/or non-physical connecting elements (e.g., wireless connection).

An energy delivery element (e.g., electrode, transducer, coil, light source, ultrasound emitter, drug delivery system, thermal module, mechanical actuator) can be controllable to generate and/or deliver stimulation paradigms with parameters that may be varied, wherein such parameters are unique to each energy modality. For electrical stimulation, adjustable parameters may include frequency, voltage, current, pulse width, intensity, duty cycle, waveform shape, polarity, burst characteristics, etc. For magnetic stimulation, adjustable parameters may include magnetic field strength, pulse repetition rate, pulse duration, coil configuration, etc. For ultrasound-based stimulation, adjustable parameters may include frequency, intensity, duty cycle, pulse duration, focal spot size, modulation scheme, etc. For optical stimulation, adjustable parameters may include wavelength, intensity, pulse duration, frequency, etc. For chemical stimulation, adjustable parameters may include drug concentration, infusion rate, release profile, carrier type, etc. For thermal stimulation, adjustable parameters may include temperature, heating/cooling rate, exposure duration, etc. For mechanical stimulation, adjustable parameters may include vibration frequency, amplitude, contact pressure, force application patterns, etc. Additionally, for any other known or future-discovered therapeutic modality, any parameters relevant to the specific energy source may be adjustable. These parameters may be modulated individually or in combination to achieve desired neuromodulatory effects.

In some embodiments, electric energy is utilized to modulate the nerve activity, and one or more energy delivery elements (or electrodes) can be controllable to generate and/or deliver stimulation paradigms that may be varied in parameters like frequency, voltage, current, pulse width, intensity, duty cycle, waveform shape, polarity burst characteristics etc. The one or more energy delivery elements or electrodes can also provide both positive current flow (e.g., from electrode/s to the tissue) and negative current flow (e.g., from tissue to the electrode/s) and/or can stop current flow from the electrode/s and/or alter the direction of current flow from the electrode/s. In some embodiments, the apparatus can include at least one electrode not only capable of generating positive and negative current but is also actively controlled to regulate current flow, stop stimulation when needed, and dynamically adjust direction based on therapy requirements. In some embodiments, energy delivery element (or electrode) is configured to deliver variable output, linear output, and short pulse-width stimulation, paired pulses and various waveforms (like square wave, sine wave, and so forth). The energy delivery element (or electrode) may be designed to have dimensions and/or size suitable for application to a particular target site of the ANS or PNS. For example, the electrode can have dimensions and/or size suitable for the vagus nerve (cranial nerve X), its branches (e.g., auricular, pharyngeal, laryngeal, cardiac, pulmonary, esophageal, and abdominal branches), the dorsal motor nucleus of the vagus, the nucleus ambiguus, intramural ganglia, and/or any combinations thereof.

The source of energy may include, for example, a battery and/or generator and/or a pulse generator that is operatively connected to one or more energy delivery elements via the controller. The source of energy may be configured to generate (or facilitate generation of) one or more stimulation paradigms. In some embodiments, the source of energy can include a battery that can be recharged using inductive coupling. Source of energy for other forms of energy, such as magnetic waves, ultrasound, optical, thermal, mechanical, microwave, radiofrequency, or any other known or future-discovered form of can also be recharged using appropriate methods. The source of energy may be positioned at various locations, including but not limited to, a position adjacent the energy delivery element (e.g., implanted near an electrode), or at a remote site within or on the subject's body. If positioned remotely, source of energy may be operatively connected to one or more energy delivery elements (e.g., electrodes, transducers) using connecting elements (e.g., wires). The remotely positioned source of energy may be an implant at a site distant from the energy delivery elements or may be positioned externally to the subject's body. In some embodiments, one or more energy delivery elements may include an integrated source of energy. The integrated source of energy may be configured to harvest energy from its surrounding environment (e.g., from the subject's biological tissues). In some embodiments, one or more energy delivery elements may be powered by positioning an external source of energy adjacent to or in contact with the subject's skin, or by incorporating an integrated source of energy, or both.

In some embodiments, the controller is configured to control one or more parameters of a stimulation paradigm (e.g., an electrical signal, magnetic wave, ultrasound). Such parameters may include, but are not limited to, signal pulse wave form, signal pulse width, signal pulse frequency, signal pulse phase, signal pulse polarity, signal pulse amplitude, signal pulse intensity, signal pulse duration, signal field strength, signal pulse repetition rate, signal duty cycle, signal focal spot size, signal modulation scheme, and/or combinations thereof. For electrical signal or electrical energy modalities, the controller may deliver various voltages and currents to one or more electrodes (energy delivery elements) to modulate the activity of a neural target within, or in communication with ANS or PNS tissue. The controller may independently manage multiple energy delivery elements (or electrodes) or activate them in various combinations to achieve stimulation (e.g., excitation) of autonomic nerve or parasympathetic nerve activity. In some embodiments, the controller may be integrated into either an external or implantable apparatus. In some embodiments, the controller can either be part of or be associated with a remotely positioned device that is in communication or can establish communication with the apparatus. In some embodiments, the controller of the remotely positioned device may be in communication with another controller, which is part of the apparatus.

In some embodiments, one or more controllers may deliver a stimulation paradigm to the energy delivery elements that may be constant, varying, and/or modulated with respect to parameters including, but not limited to, current, voltage, frequency, pulse width, waveform, cycle, amplitude, etc.

In some embodiments, the current may range from approximately 0.001 to 100,000 microamperes (μA), where 1 microampere (μA) is equal to 10⁻⁶ amperes. This range is intended to encompass the full spectrum of current levels used in nerve modulation (or neuromodulation) applications. Currently, currents around 0.001 microampere (μA) are used for single-neuron stimulation, while 100,000 μA represents the highest known current used for transcutaneous applications. Future advancements that may extend this range, either by lowering the minimum or increasing the maximum current, are explicitly considered within the scope of this disclosure.

In some embodiments, the voltage may range from approximately 0.01 millivolt (mV) to 50 volts (V), where 1 millivolt (mV) is equal to 103 volts. This range is intended to encompass the full spectrum of voltage levels used in nerve modulation applications. Currently, electrophysiology research suggests that neurons can be activated with voltages as low as 0.01 millivolt (mV), while 50 volts (V) represents the highest known voltage used for spinal cord stimulation and vagus nerve stimulation. Future advancements that may extend this range, either by lowering the minimum or increasing the maximum voltage, are explicitly considered within the scope of this disclosure.

In some embodiments, the frequency for electrical stimulation may range from approximately 0.01 Hz to 100 kHz, where 1 kHz (Kilohertz) is equal to 10³ Hz. This range is intended to encompass the full spectrum of frequency range used in electrical nerve modulation applications. Currently, extremely low-frequency (ELF) stimulation as low as 0.01 Hz is used for slow-wave electrical stimulation in sleep studies, while 100 kHz represents the highest known frequency used for nerve conduction block to inhibit nerve activity without damaging the nerve. Future advancements that may extend this range, either by lowering the minimum or increasing the maximum frequency, are explicitly considered within the scope of this disclosure. Additionally, other energy modalities, including but not limited to magnetic, radiofrequency, ultrasound, or optical, may each have their own distinct frequency ranges, which would be recognized by a person skilled in the art. This disclosure explicitly encompasses the full spectrum of frequency ranges for each of these modalities, including any future advancements that modify these ranges.

In some embodiments, the pulse-width may range from approximately 1 to 10,000 microseconds (μs), where 1 microsecond (μs) is equal to 10⁻⁶ seconds. This range is intended to encompass the full spectrum of pulse-width used in nerve modulation applications. Currently, pulse-width around 1 microsecond (μs) is used for nerve conduction block, while 10,000 microseconds (μs) represents the highest known pulse-width used for electrical muscle stimulation (EMS). Future advancements that may extend this range, either by lowering the minimum or increasing the maximum pulse-width, are explicitly considered within the scope of this disclosure.

In some embodiments, the stimulation signal, which may or not be electrical, may be oscillatory. The type of stimulation may vary and involve different waveforms known to the skilled artisan and/or novel waveforms generated by artificial intelligence (AI) algorithm/s.

In some embodiments, to modulate activity (e.g., excitation) in a neural target comprising a target site of the ANS or PNS, parameters of the stimulation paradigm (e.g., of electrical stimulation) may include, for example, voltage ranging from approximately 0.01 millivolt (mV) to 5 volt (V) or more, a current ranging from approximately 0.001 to 50 milliamperes (mA), and a frequency ranging from approximately 10 Hz to about 100 Hz. Anecdotal evidence in a peer-reviewed study suggests that electrical stimulation with frequency below 10 Hz may induce hot flashes (symptom of menopause). A highly skilled person in the art would recognize that electrical stimulation frequencies below 10 Hz and above 10 Hz are known to have diametrically opposite effects, therefore, frequencies above 10 Hz may be used to counteract or mitigate hot flashes. Furthermore, a person skilled in the art would recognize that by modulating stimulation parameters other than frequency in a novel manner, it may be possible to counteract or mitigate hot flashes at 10 Hz or less as well, and such electrical stimulation paradigms are considered within the scope of this disclosure. In some embodiments, an electrical stimulation paradigm can have a frequency range of 10,000 Hz or more (high frequency stimulation) to effectively modulate nerve activity. In some embodiments, AC and/or pure DC voltages may be utilized. In some embodiments, pulse-width may range from approximately 1 to about 10,000 microseconds (μs) or more. The electrical stimulation paradigm may be applied for at least about 1 microsecond or more, for example, 20 milliseconds or one second or several seconds. In some embodiments, the energy (e.g., electric, magnetic, ultrasound), in the form of stimulation paradigm may be applied for 1 minute or several minutes or 30 minutes or more. In some embodiments, the energy (e.g., electric, magnetic, ultrasound), in the form of stimulation paradigm may be applied for as long as about 1 hour or several hours or more (e.g., 24 hours or 72 hours or more), as needed to achieve a therapeutic effect.

In some embodiments, the energy delivery elements or electrodes may be configured as monopolar, bipolar or multipolar. In some embodiments, to minimize potential immune responses triggered by the subject's body against the apparatus and to reduce potential damage (to apparatus' components), the energy delivery elements (e.g., electrodes), connecting elements (e.g., wires), and/or other components of the apparatus may be fabricated from inert, biocompatible materials. In some embodiments, an apparatus may comprise one or more multipolar energy delivery elements or electrodes with multiple exposed contacts to enhance therapeutic efficacy and more precise targeting of the indication.

In some embodiments, the apparatus includes a controller which may incorporate at least one microprocessor operable via suitable software. Additionally, controller may have other components including but not limited to, a digital signal processor (DSP), a field-programmable gate array (FPGA), an analog-to-digital converter (ADC), a digital-to-analog converter (DAC), memory storage, power management circuitry, wireless communication modules, and/or biofeedback sensors. In some embodiments, the controller may be configured to record data from a nerve or neural target of the subject and store it either locally (e.g., on the apparatus) or remotely (e.g., on a cloud-based system). In some embodiments, the controller may be configured to analyze the recorded data and/or dynamically adjust the parameters of stimulation paradigm. In some embodiments, the controller may be configured to apply one or more stimulation paradigms to the energy delivery element/s (or electrode/s) in response to changes in the neural activity (e.g., excitability) of a target site of the ANS or PNS. Delivery of such stimulation paradigms may be triggered if one or more recorded data points fall below or exceed a predefined threshold or a threshold inferred by an artificial intelligence (AI) algorithm using historical and/or real-time data analysis.

In some embodiments, the apparatus may be pre-programmed with a specific stimulation paradigm whose parameters may include, but is not limited to, frequency, current levels, waveform and pulse width. In some embodiments, the parameters of the stimulation paradigm may be adjustable remotely, allowing for remote modifications to any parameters of the stimulation paradigm without necessitating the removal and/or physical repositioning of the apparatus from its target site. Controlling the stimulation paradigm parameters remotely may be achieved using, for example, via an implanted radiofrequency (RF) receiver coupled to an external transmitter, telemetry with an implanted power source, inductive coupling, ultrasonic energy transfer, optical-based communication, Bluetooth, near-field communication (NFC), cloud-based control mechanisms, or any other wired or wireless communication modality, whether presently available or developed in the future. In some embodiments, stimulation parameters may be pre-programmed such that a stimulation paradigm is automatically delivered to a target site within or in communication with the ANS or PNS at a predefined frequency, current, pulse width, waveform, etc. For example, stimulation parameters may be pre-programmed to apply a stimulation paradigm to a target site of the ANS or PNS between a specified time interval, such as from 10:00 PM to 11:00 PM, or during any other time window, and/or for any duration (e.g., minutes, hours, or longer). In some embodiments, one or more parameters of the stimulation paradigm may be adjustable by the subject without requiring supervision and/or instructions from an expert, including but not limited to a clinician, physician, or OB-GYN.

In some embodiments, the apparatus can be configured for intravascular placement, which refers to the positioning of the apparatus within (or even outside) a blood vessel, such as an artery, vein, or microvascular structure, to enable targeted stimulation, neuromodulation, sensing, or physiological monitoring. In some embodiments, the apparatus can be configured for intravascular placement at a site that is adjacent, directly adjacent, or in proximity to a target site of the ANS or PNS, for e.g., one or more sites of the vagus nerve (cranial nerve X), its branches (e.g., auricular, pharyngeal, laryngeal, cardiac, pulmonary, esophageal, and abdominal branches), the dorsal motor nucleus of the vagus, the nucleus ambiguus, intramural ganglia, and/or any combinations thereof.

In some embodiments, the apparatus may be configured for non-invasive (e.g., transcutaneous) modulation of a nerve or neural target. In some embodiments, non-invasive (e.g., transcutaneous) modulation of a nerve or neural target may involve positioning an apparatus on, near, or in proximity to the skin surface of the subject to deliver a stimulation paradigm. The stimulation paradigm may include, but is not limited to, an electrical signal, ultrasound signal, optical signal, electromagnetic field, and/or other energy-based modalities. The stimulation may be delivered to a target site within or in communication with the ANS or PNS, including neural targets associated with menopause and/or its associated symptoms. Such target sites may include, but are not limited to, one or more locations along the main trunk, branches, or associated vascular structures of the vagus nerve. Such targets may also include any tissues of the parasympathetic nervous system (PNS) including, but are not limited to, neurons, axons, fibers, tracts, nerves, plexus, afferent plexus fibers, efferent plexus fibers, ganglia, pre-ganglionic fibers, post-ganglionic fibers, the vagus nerve (cranial nerve X), its branches (e.g., auricular, pharyngeal, laryngeal, cardiac, pulmonary, esophageal, and abdominal branches), the dorsal motor nucleus of the vagus, the nucleus ambiguus, intramural ganglia, and/or any combinations thereof. The term ‘target site’ is defined as any site within, or in communication with, the ANS or PNS where modulating the nerve activity produces a therapeutic outcome for menopause and/or its associated symptoms.

In some embodiments, transcutaneous modulation of a nerve or neural target may also include partially transcutaneous or minimally invasive methods, for e.g., employing a sharp component to pierce the epidermis. In some embodiments, the sharp component may be an extraneural or intraneural energy delivery element (e.g., electrode or microelectrode). In some embodiments, a minimally invasive approach may be used, where one or more intraneural energy delivery elements (e.g., electrode or microelectrodes) are employed to enhance efficacy, improve precision targeting and/or reduce paresthesia (or stimulation-induced sensations). In some embodiments, a minimally invasive approach may be used, wherein one or more intraneural energy delivery elements (e.g., electrode or microelectrodes) are employed, thereby allowing the range of stimulation parameters to be similar to those used in invasive interventions. Intraneural energy delivery elements enable stimulation or neuromodulatory effects to be achieved with lower-intensity stimulation paradigms, requiring less energy or reduced parameter values.

In some embodiments, the stimulation paradigm used for non-invasive (e.g., transcutaneous) modulation of the nerve or neural target may be constant, varying, and/or modulated with respect to parameters including, but not limited to, current, voltage, frequency, pulse width, waveform, cycle, amplitude, etc. In some embodiments, the current may range from approximately 0.001 to 100,000 microamperes (μA), where 1 microampere (μA) is equal to 10⁻⁶ amperes. This range is intended to encompass the full spectrum of current levels used in nerve modulation (neuromodulation) applications. Future advancements that may extend this range, either by lowering the minimum or increasing the maximum current, are explicitly considered within the scope of this disclosure. In some embodiments, the voltage may range from approximately 0.01 millivolt (mV) to 50 volts (V), where 1 millivolt (mV) is equal to 10³ volts. This range is intended to encompass the full spectrum of voltage levels used in nerve modulation applications. Future advancements that may extend this range, either by lowering the minimum or increasing the maximum voltage, are explicitly considered within the scope of this disclosure. In some embodiments, the frequency for electrical stimulation may range from approximately 0.01 Hz to 100 kHz, where 1 kHz (Kilohertz) is equal to 10³ Hz. This range is intended to encompass the full spectrum of frequency range used in electrical nerve modulation applications. Future advancements that may extend this range, either by lowering the minimum or increasing the maximum frequency, are explicitly considered within the scope of this disclosure. Additionally, other energy modalities, including but not limited to magnetic, radiofrequency, ultrasound, or optical, may each have their own distinct frequency ranges, which would be recognized by a person skilled in the art. This disclosure explicitly encompasses the full spectrum of frequency ranges for each of these modalities, including any future advancements that modify these ranges. In some embodiments, the pulse-width may range from approximately 1 to 10,000 microseconds (μs), where 1 microsecond (μs) is equal to 10⁻⁶ seconds. This range is intended to encompass the full spectrum of pulse-width used in nerve modulation applications. Future advancements that may extend this range, either by lowering the minimum or increasing the maximum pulse-width, are explicitly considered within the scope of this disclosure. In some embodiments, the stimulation signal, which may or not be electrical, may be oscillatory. The type of stimulation may vary and involve different waveforms known to the skilled artisan and/or novel waveforms generated by artificial intelligence (AI) algorithm/s.

In some embodiments, the apparatus can be configured for implantation within the body of a subject. Such implantation of an apparatus may be performed using a percutaneous and/or minimally invasive and/or invasive approaches.

In accordance with aspects of the present disclosure, an exemplary method for treating menopause and associated symptoms by modulating nerve activity is provided. The method generally includes a framework (or approach, modality, or process) that may be employed to practice the present disclosure. In some aspects, the method may involve modulating the activity of a nerve or neural target at one or more sites within the autonomic nervous system (ANS) or parasympathetic nervous system (PNS), where such nerve or neural target is directly or indirectly associated with menopause and/or its associated symptoms. The term ‘sites within the autonomic nervous system (ANS) or parasympathetic nervous system (PNS)’ refers to any site within, or in communication with, the ANS or PNS where modulating the nerve activity produces a therapeutic outcome for menopause and/or associated symptoms. The site (or sites), as described herein, may include, but are not limited to, one or more locations along the main trunk, branches, or associated vascular structures of the vagus nerve. The site (or sites), as described herein, may also include any tissues of the ANS or PNS including, but not limited to, neurons, axons, fibers, tracts, nerves, plexus, afferent plexus fibers, efferent plexus fibers, ganglia, pre-ganglionic fibers, post-ganglionic fibers, the vagus nerve (cranial nerve X), its branches (e.g., auricular, pharyngeal, laryngeal, cardiac, pulmonary, esophageal, and abdominal branches), the dorsal motor nucleus of the vagus, the nucleus ambiguus, intramural ganglia, and/or any combinations thereof.

The present disclosure provides the scope and definition of menopause and associated symptoms. In some aspects, subjects, individuals or patients who may benefit from the present disclosure can be diagnosed with, or are suspected of having, menopause and/or one or more associated symptoms and/or one or more related or unrelated medical indications.

In some aspects, a method for treating menopause and associated symptoms by modulating nerve activity can be practiced by placing an apparatus in communication (e.g., electrical, magnetic, electromagnetic, acoustic, optical, and/or other energy-based modalities) with a target site of the autonomic nervous system (e.g., autonomic nerve target) or parasympathetic nervous system (e.g., parasympathetic nerve target), where the target site is directly or indirectly associated with the menopause and/or its associated symptoms. This method can be practiced via a transcutaneous, or transdermal, or percutaneous, or intravascular, or invasive approach. In some aspects, it may be desirable to modulate nerve activity by placing an apparatus into electrical communication with only one specific parasympathetic nervous tissue or nerve structure, for e.g., auricular branch of the vagus nerve (ABVN) located in conch area of the ear (e.g., cymba concha), where certain advantages, including but not limited to therapeutic effects and better UX (user experience), may be realized.

Once the apparatus is placed, the apparatus may be activated to deliver a stimulation paradigm to a target site of the ANS or PNS. In some aspects, delivering a stimulation paradigm to a target site of the ANS or PNS can prevent menopause or one or more of its associated symptoms by either increasing or decreasing the excitability of target site (or nerve) from a predefined threshold or a threshold inferred by an artificial intelligence (AI) algorithm. In some aspects, delivery of a stimulation paradigm to a target site of the ANS or PNS can decrease a sign and/or symptom associated with the menopause by either increasing or decreasing the excitability of the target site (or nerve) from a predefined threshold or a threshold inferred by an artificial intelligence (AI) algorithm. In some aspects, the apparatus may be activated at the onset of one or more symptoms associated with the menopause. In some aspects, the apparatus may be activated intermittently or continuously to either completely eliminate or reduce the frequency and/or intensity and/or effect of one or more symptoms of menopause.

Delivery of stimulation paradigm to a target site of the ANS or PNS, e.g., vagus nerve, can influence central autonomic regulation, nerve excitability, neurotransmitter levels (e.g., acetylcholine, norepinephrine, serotonin, gamma-aminobutyric acid (GABA)), synaptic plasticity, neuroplasticity, and neuronal activity at one or more associated target tissues. These target tissues may include, but are not limited to, the hypothalamus and median preoptic nucleus (MnPO), which are key regions involved in thermoregulation, neuroendocrine control, and autonomic homeostasis. In some aspects, delivery of a stimulation paradigm (e.g., an electrical signal, ultrasound signal, or electromagnetic signal) to a target site of the ANS or PNS, e.g., vagus nerve, can increase vagal excitability, thereby facilitating reliable neural signaling, enhanced information transfer (e.g., temperature information), and activity-dependent neuroplasticity in vagal projection sites. This modulation may result in adaptive changes in synaptic strength, activity-dependent plasticity (including but not limited to long-term potentiation (LTP) or depression (LTD)), and circuit-level reorganization, leading to sustained improvements in neuroendocrine thermoregulation, cardiovascular control, inflammatory responses, cognitive function, mood regulation, metabolic processes, and other physiological functions associated with menopause and its associated symptoms.

Prior applications of stimulating the vagus nerve-which is part of the parasympathetic nervous system (PNS), a subdivision of the autonomic nervous system (ANS)-have primarily focused on treating neurological and autonomic conditions such as epilepsy, depression, and inflammation-related disorders. However, vagus nerve stimulation (VNS) has not been previously explored as a therapy for menopause and its associated symptoms. Menopause-related autonomic c dysfunction, neuroendocrine changes, thermoregulatory instability, and inflammatory imbalances have been widely documented. Additionally, preclinical evidence suggests that estrogen deficiency results in reduced excitability of a sexually dimorphic subset of myelinated vagal afferents, an effect that has not been previously investigated in the context of treating menopause, hot flashes, and/or other symptoms of menopause. Unlike prior VNS applications that focus on broad autonomic or inflammatory modulation, the present disclosure establishes a novel use of VNS for addressing menopause and its associated symptoms, including but not limited to hot flashes, night sweats, sleep disturbances, cognitive dysfunction, cardiovascular irregularities, and metabolic shifts. By leveraging VNS to compensate for menopause-associated vagal hyporesponsiveness, this disclosure provides a new therapeutic approach distinct from existing pharmacological and non-pharmacological interventions. One skilled in the art can titrate the degree of modulation (e.g., increase excitability) for vagus nerve or other neural tissue in parasympathetic nervous system, for example, based upon the nature and severity of the menopause and/or its associated symptom.

A separate approach that has been explored for treating menopausal symptoms, particularly hot flashes, is Stellate Ganglion Block (SGB). SGB was incidentally identified as a potential therapy for hot flashes when patients in clinical trials for unrelated indications reported a reduction in hot flashes. Although clinical evidence supporting its efficacy remains preliminary, the role of sympathetic activity modulation in hot flashes is widely recognized in scientific literature, including reports by the North American Menopause Society. Currently, SGB for hot flashes or other indications is primarily performed via anesthetic injection into the stellate ganglion to block sympathetic activity. However, those skilled in the art recognize that such interventions can be replicated using device-based neuromodulation approaches. A US patent (US20170281937) was granted nearly a decade ago for a device-based intervention aimed at modulating sympathetic activity for treating hot flashes. This disclosure explicitly excludes neuromodulation approaches that replicate or rely on SGB mechanisms or sympathetic activity modulation. Instead, this disclosure focuses exclusively on neuromodulation of neural targets within or in communication with the ANS or PNS, representing a distinct therapeutic approach that is not based on sympathetic modulation and has not been previously applied to menopause and associated symptoms. This disclosure includes neural targets within or in communication with the ANS or PNS, as they have the potential to deliver therapeutic effects for menopause and its associated symptoms. The ANS and PNS project to multiple brain regions associated with menopausal symptoms, thereby, its stimulation induces neuroplasticity and influences neural signaling to achieve therapeutic outcomes for menopause.

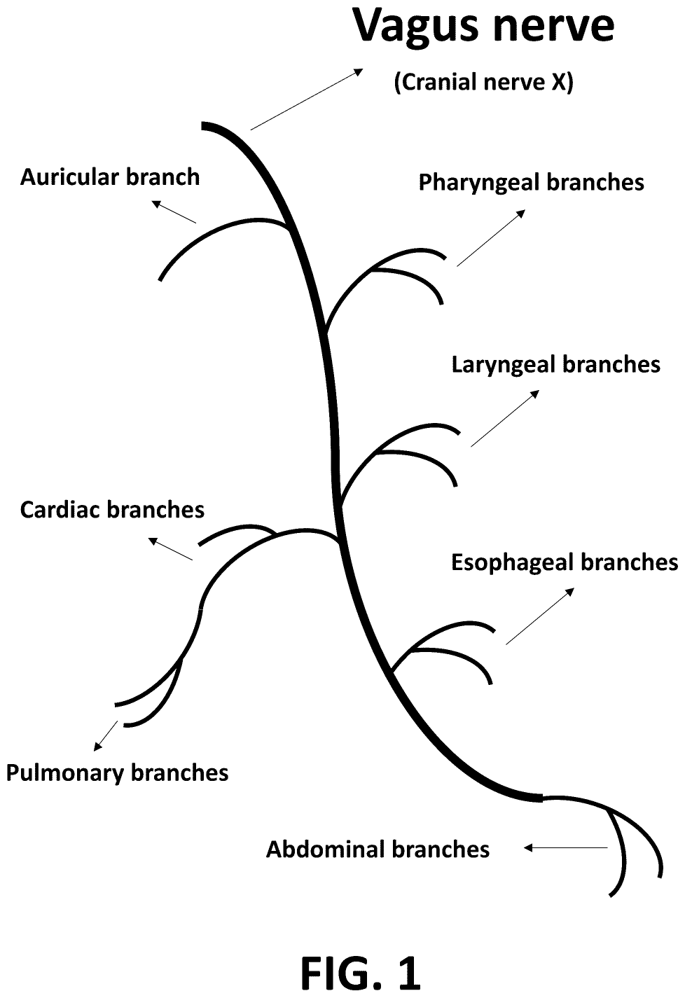

FIG. 1 is a schematic illustration showing the vagus nerve (cranial nerve X) and its major branches, including the auricular, pharyngeal, laryngeal, cardiac, esophageal, pulmonary, and abdominal branches. These branches represent potential target sites for one or more apparatuses, systems and methods described in the present disclosure.

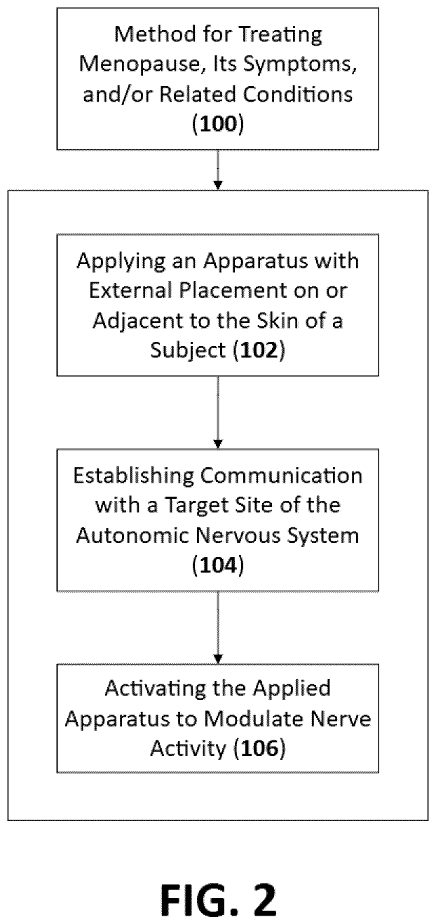

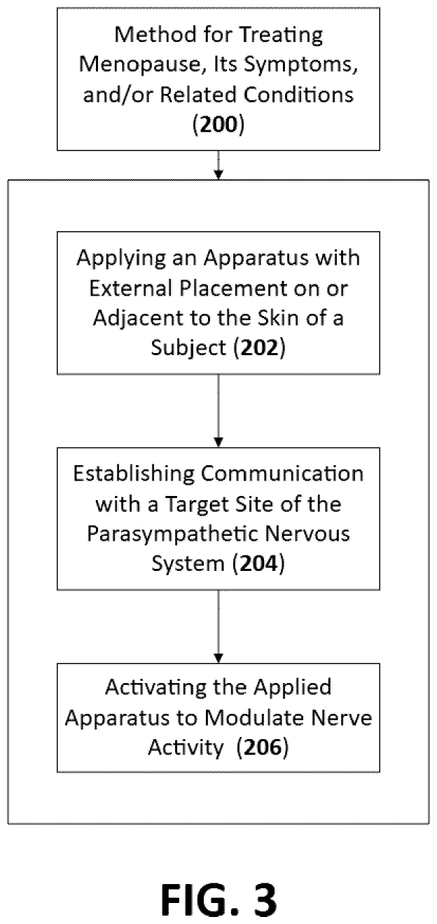

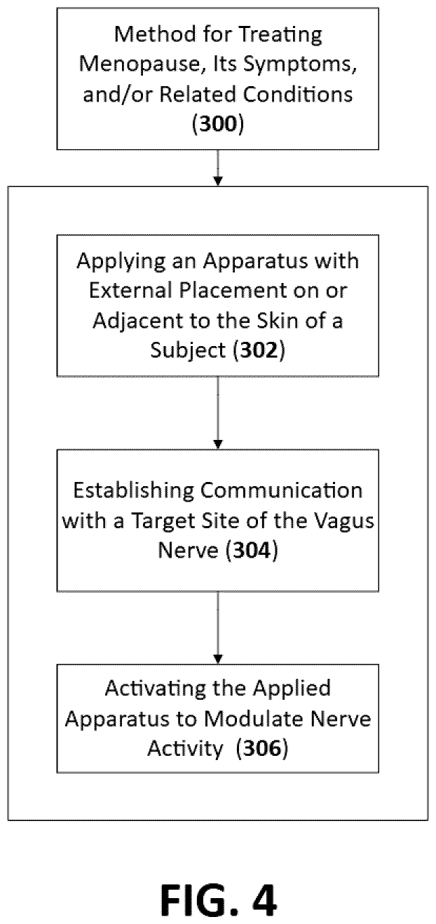

FIGS. 2-4 illustrate exemplary methods 100, 200, 300 for treating menopause, its associated symptoms (e.g., hot flashes), and/or related conditions via a non-invasive (e.g., transcutaneous) approach. For exemplary methods 100, 200, 300, steps 102, 202, 302 correspond, respectively, which involve applying an apparatus externally on or adjacent to the skin (e.g., cymba concha) of a subject. In some aspects, exemplary method 100 establishes communication (using electrical, magnetic, ultrasound, optical, and/or other energy-based modalities) with a target site of the autonomic nervous system (104). In some aspects, method 200 establishes communication (using electrical, magnetic, ultrasound, optical, and/or other energy-based modalities) with a target site of the parasympathetic nervous system (204). In some aspects, method 300 establishes communication (using electrical, magnetic, ultrasound, optical, and/or other energy-based modalities) with a target site of the vagus nerve (304). A target site, as described herein, may be directly or indirectly associated with the menopause and/or its associated symptoms, and include sites, including but not limited to, neurons, axons, fibers, tracts, nerves, plexus, afferent plexus fibers, efferent plexus fibers, ganglia, pre-ganglionic fibers, post-ganglionic fibers, the vagus nerve (cranial nerve X), its branches (e.g., auricular, pharyngeal, laryngeal, cardiac, pulmonary, esophageal, and abdominal branches), the dorsal motor nucleus of the vagus, the nucleus ambiguus, intramural ganglia, and/or any combinations thereof. For exemplary methods 100, 200, 300, steps 106, 206, 306 correspond, respectively, which involve activating the applied apparatus or systems to deliver one or more stimulation paradigms to modulate the nerve activity. In some aspects, a method may involve placing an apparatus in communication with the auricular branch of vagus nerve (ABVN) which is found in conch area of the ear (e.g., cymba concha).

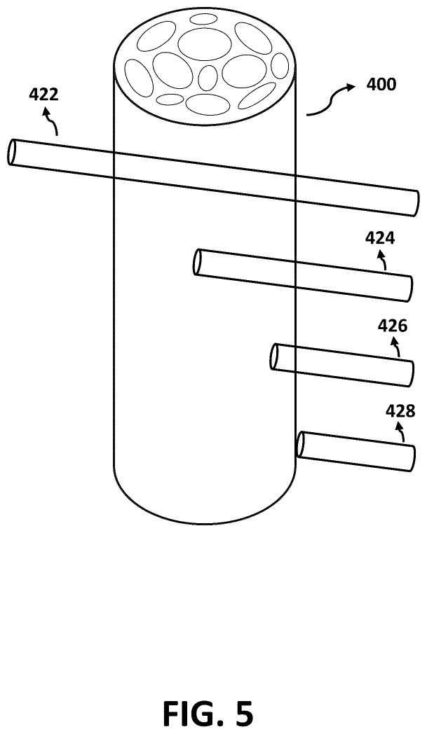

FIG. 5 shows a perspective view of several exemplary energy delivery elements 422, 424, 426, 428 for treating menopause, its symptoms, and/or related conditions in a subject by modulating activity of a nerve 400. In some embodiments, an apparatus may include one or more intraneural energy delivery elements (422, 424 and/or 426) in communication with a target site of the autonomic nervous system (ANS) or parasympathetic nervous system (PNS). In some embodiments, an apparatus may include one or more intraneural energy delivery elements configured to fully penetrate a nerve, by entering at one spot and exiting at another, such as element 422. The entry and exit points may be positioned at any two locations on the nerve. In some embodiments, an apparatus may include one or more intraneural energy delivery elements configured to partially penetrate a nerve, entering at one spot but not exiting, such as elements 424 and 426. In some embodiments, an apparatus may include one or more extraneural energy delivery elements 428 in communication with a target site of the autonomic nervous system (ANS) or parasympathetic nervous system (PNS). In some embodiments, an apparatus may include one or more extraneural energy delivery elements (e.g., electrodes) placed adjacent to a target site or nerve 400, where the energy delivery element may be invasive, minimally invasive, or non-invasive (e.g., transcutaneous).

In some aspects, the method may involve use of one or more intraneural energy delivery elements (e.g., 422 and/or 424 and/or 426) to deliver a stimulation paradigm to a target site of the autonomic nervous system (ANS) or parasympathetic nervous system (PNS), where the target side is auricular branch of vagus nerve (ABVN). Traditional peripheral nerve stimulation platforms typically avoid intraneural approaches due to associated risks, despite the potential for significantly higher efficacy. However, certain peripheral nerve structures (e.g., in ANS or PNS), such as distal or superficial or terminal branches of the ABVN within the ear (e.g., cymba concha), present a unique opportunity for intraneural energy delivery. These sites provide access to the nerve or neural targets (in ANS or PNS) suitable for intraneural stimulation while maintaining an almost non-invasive (or minimally invasive) profile and low risk.

The cymba concha has been a site for auricular piercings for centuries, and it is highly likely that some of these piercings inadvertently penetrated small terminal branches of the ABVN. However, there are no known reports of complications directly attributable to nerve penetration in this region. This suggests that targeted penetration of distal or superficial or terminal branches of peripheral nerves, particularly within autonomic or parasympathetic nervous structures, presents an innovative, feasible, and low-risk approach to neuromodulation. Accordingly, such methods and embodiments fall within the scope of this disclosure.

In some aspects and embodiments, an apparatus may include one or more intraneural energy delivery elements (422, 424 and/or 426) configured to be in communication with a target site of the ANS or PNS, where the target site is distal or superficial or terminal branches of a nerve (or neural target). In some aspects and embodiments, an apparatus may include one or more intraneural energy delivery elements (422, 424 and/or 426) configured to be in communication with a target site of the ANS or PNS, where the target site is distal or superficial or terminal branches of vagus nerve. In some aspects and embodiments, an apparatus may include one or more intraneural energy delivery elements (422, 424 and/or 426) configured to be in communication with a target site of the ANS or PNS, where the target site is distal or superficial or terminal branches of auricular branch of vagus nerve (ABVN).

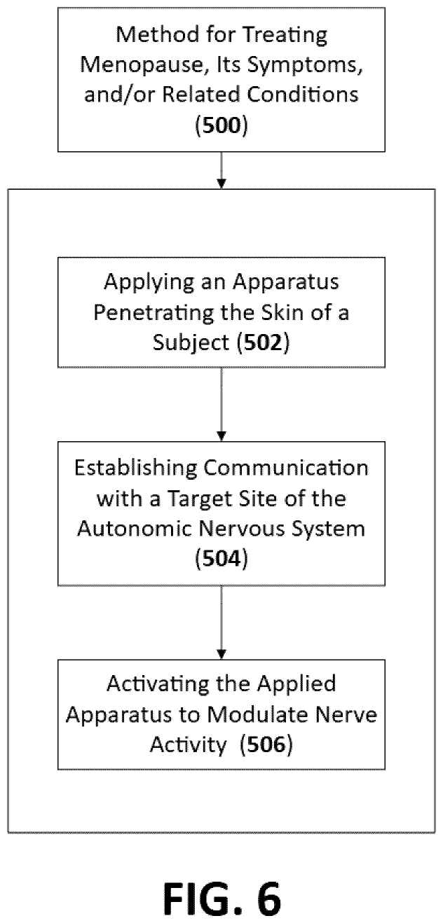

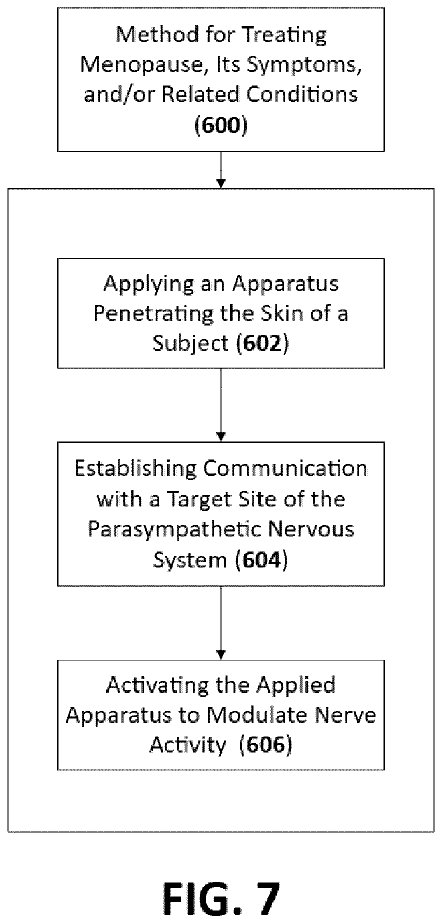

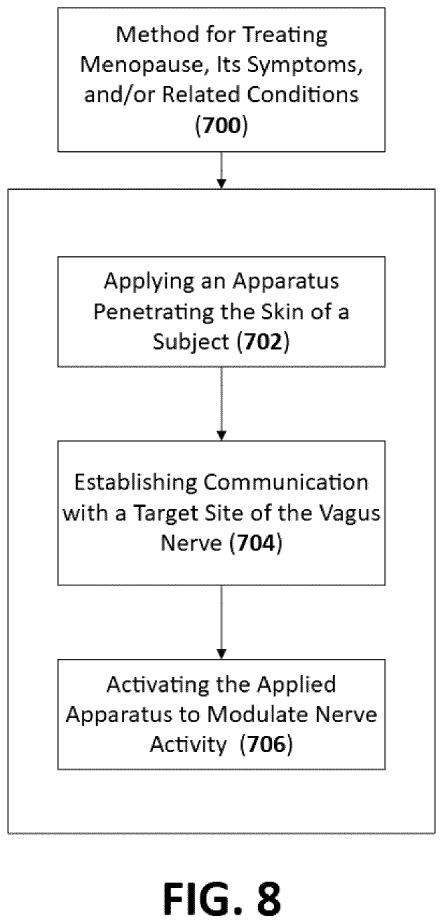

FIGS. 6-8 illustrate exemplary methods 500, 600, 700 for treating menopause, its associated symptoms (e.g., hot flashes), and/or related conditions via a minimally invasive (e.g., percutaneous) approach. For exemplary methods 500, 600, 700, steps 502, 602, 702 correspond, respectively, which involve applying an apparatus penetrating the skin (e.g., at cymba concha) of a subject. In some aspects, exemplary method 500 establishes communication (using electrical, magnetic, ultrasound, optical, and/or other energy-based modalities) with a target site of the autonomic nervous system (504). In some aspects, method 600 establishes communication (using electrical, magnetic, ultrasound, optical, and/or other energy-based modalities) with a target site of the parasympathetic nervous system (604). In some aspects, method 700 establishes communication (using electrical, magnetic, ultrasound, optical, and/or other energy-based modalities) with a target site of the vagus nerve (704). A target site, as described herein, may be directly or indirectly associated with the menopause and/or its associated symptoms, and include sites, including but not limited to, neurons, axons, fibers, tracts, nerves, plexus, afferent plexus fibers, efferent plexus fibers, ganglia, pre-ganglionic fibers, post-ganglionic fibers, the vagus nerve (cranial nerve X), its branches (e.g., auricular, pharyngeal, laryngeal, cardiac, pulmonary, esophageal, and abdominal branches), the dorsal motor nucleus of the vagus, the nucleus ambiguus, intramural ganglia, and/or any combinations thereof. For exemplary methods 500, 600, 700, steps 506, 606, 706 correspond, respectively, which involve activating the applied apparatus or systems to deliver one or more stimulation paradigms to modulate the nerve activity. In some aspects, a method may involve an apparatus penetrating the skin of a subject to establish communication with the auricular branch of vagus nerve (ABVN) which is found in cymba concha of the ear. The apparatus may use one or more intraneural and/or extraneural energy delivery elements to establish communication with the ABVN.

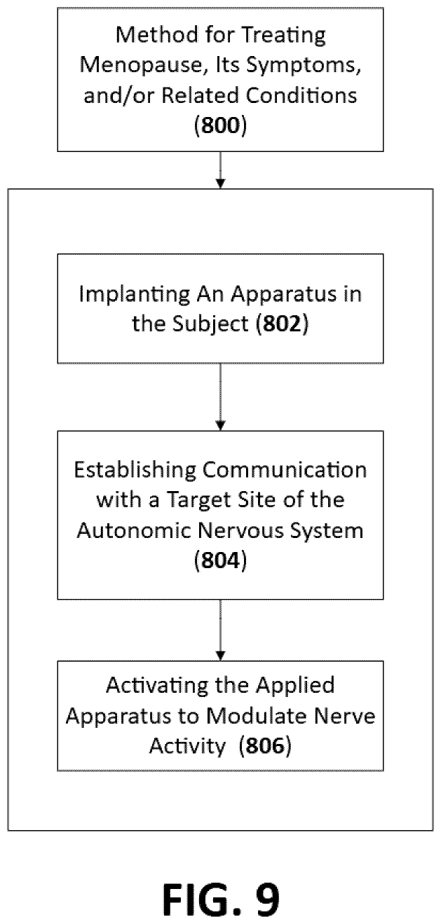

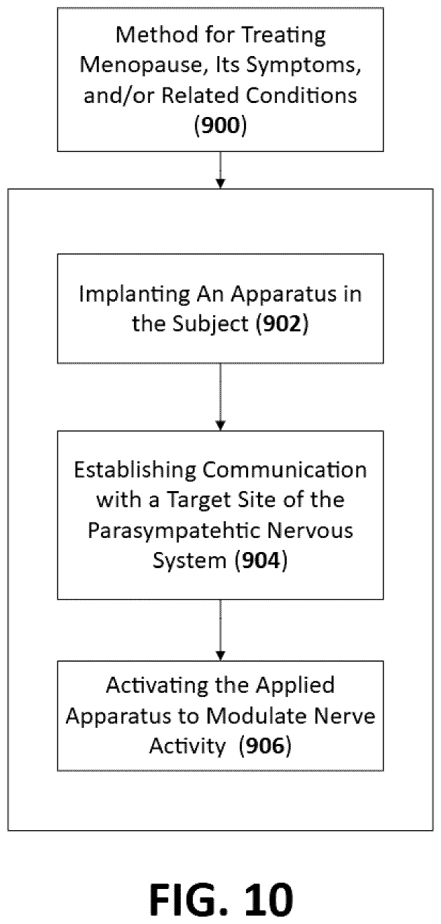

FIGS. 9-10 illustrate exemplary methods 800, 900 for treating menopause, its associated symptoms (e.g., hot flashes), and/or related conditions via an invasive (e.g., implant) approach. For exemplary methods 800, 900, steps 802, 902, correspond, respectively, which involve implanting an apparatus in the Subject. In some aspects, exemplary method 800 establishes communication (via electrical, magnetic, ultrasound, optical, and/or other energy-based modalities) with a target site of the autonomic nervous system (804). In some aspects, method 900 establishes communication (via electrical, magnetic, ultrasound, optical, and/or other energy-based modalities) with a target site of the parasympathetic nervous system (904) e.g., vagus nerve. A target site, as described herein, may be directly or indirectly associated with the menopause and/or its associated symptoms, and include sites, including but not limited to, neurons, axons, fibers, tracts, nerves, plexus, afferent plexus fibers, efferent plexus fibers, ganglia, pre-ganglionic fibers, post-ganglionic fibers, the vagus nerve (cranial nerve X), its branches (e.g., auricular, pharyngeal, laryngeal, cardiac, pulmonary, esophageal, and abdominal branches), the dorsal motor nucleus of the vagus, the nucleus ambiguus, intramural ganglia, and/or any combinations thereof. For exemplary methods 800, 900, steps 806, 906 correspond, respectively, which involve activating the applied apparatus or systems to deliver one or more stimulation paradigms to modulate the nerve activity. In some aspects, a method may involve implanting an apparatus in the main trunk of vagus which is found in the neck. The apparatus may use one or more intraneural and/or extraneural energy delivery elements to establish communication with vagus nerve.

In some aspects, the apparatus may be programmed so that the stimulation paradigm is automatically delivered to the target site of the ANS or PNS or Vagus nerve at pre-defined stimulation parameters (e.g., frequency, current). In some aspects, subject may trigger activation of the apparatus, e.g., by actuating a button of the apparatus. In some aspects, subject may trigger activation of the apparatus in response to onset of a symptom (e.g., hot flashes). The stimulation paradigm may be delivered for necessary time and intensity (related to amount of energy & value of stimulation parameters) to effectively counteract or mitigate or eliminate the symptoms/s (e.g., hot flashes).

While exemplary embodiments and aspects have been described herein, it is expressly noted that these embodiments and aspects should not be construed as limiting, but rather that additions and modifications to what is expressly described herein also are included within the scope of the invention. Moreover, it is to be understood that the features of the various embodiments and aspects described herein are not mutually exclusive and can exist in various combinations and permutations, even if such combinations or permutations are not made express herein, without departing from the spirit and scope of the invention.

From the description of the present disclosure, those skilled in the art will perceive improvements, changes and modifications. Such improvements, changes, and modifications are within the skill of those in the art and are intended to be covered by the appended claims.

1. A method for treating menopause and associated symptoms in a subject, the method comprising the steps of:
externally placing a neuromodulation device, wherein at least one of its components establishes communication with a target site of the autonomic nervous system of the subject; and
activating the neuromodulation device, wherein the neuromodulation device is configured to deliver a stimulation paradigm to the target site of the autonomic nervous system; and
thereby modulating the nerve activity to induce an effect in the subject; and
wherein the induced effect excludes change in sympathetic activity induced by an electrical energy source; and
thereby treating menopause and associated symptoms.

2. The method of claim 1, wherein the target site of autonomic nervous system is vagus nerve.

3. The method of claim 1, wherein at least one of the components of the neuromodulation device is configured to deliver a stimulation paradigm to the target site of the autonomic nervous system using a transcutaneous approach.

4. The method of claim 1, target site of autonomic nervous system is a superficial neural target and the neuromodulation device comprises at least one intraneural energy delivery element.

5. The method of claim 1, wherein the neuromodulation device is configured to automatically deliver a stimulation paradigm to the target site of autonomic nervous system.

6. The method of claim 1, wherein the neuromodulation device is configured to deliver a stimulation paradigm where the parameters of stimulation paradigm are pre-defined.

7. The method of claim 1, wherein the neuromodulation device is configured to deliver a stimulation paradigm where the parameters of stimulation paradigm are defined by artificial intelligence algorithm.

8. The method of claim 7, wherein the parameters of stimulation paradigm are modified in real-time.

9. A method for treating menopause and associated symptoms in a subject, the method comprising the steps of:
externally placing a neuromodulation device, wherein at least one of its components establishes communication with a target site of the parasympathetic nervous system of the subject; and
activating the neuromodulation device, wherein the neuromodulation device is configured to deliver a stimulation paradigm to the target site of the parasympathetic nervous system; and
thereby modulating the nerve activity to induce an effect in the subject and thereby treating menopause and associated symptoms.

10. The method of claim 9, wherein the target site of parasympathetic nervous system is vagus nerve.

11. The method of claim 9, wherein the neuromodulation device is configured to deliver a stimulation paradigm to the target site of the parasympathetic nervous system using a transcutaneous approach.

12. The method of claim 9, wherein the target site of parasympathetic nervous system is a superficial neural target and the neuromodulation device comprises at least one intraneural energy delivery element.

13. The method of claim 9, wherein the neuromodulation device is configured to automatically deliver a stimulation paradigm to the target site of parasympathetic nervous system.

14. The method of claim 9, wherein the neuromodulation device is configured to deliver a stimulation paradigm where the parameters of stimulation paradigm are pre-defined.

15. The method of claim 9, wherein the neuromodulation device is configured to deliver a stimulation paradigm where the parameters of stimulation paradigm are defined by artificial intelligence algorithm.

16. The method of claim 15, wherein the parameters of stimulation paradigm are modified in real-time.

17. A method for treating menopause and associated symptoms in a subject, the method comprising the steps of:
externally placing a neuromodulation device, wherein at least one of its components establishes electrical communication with a target site of the parasympathetic nervous system of the subject; and
activating the neuromodulation device, wherein the neuromodulation device is configured to deliver a stimulation paradigm to the target site of the parasympathetic nervous system; and
thereby modulating the nerve activity to induce an effect in the subject and thereby treating menopause and its associated symptoms.

18. The method of claim 17, wherein the target site of parasympathetic nervous system is vagus nerve.

19. The method of claim 18, wherein the neuromodulation device is configured to deliver a stimulation paradigm at a frequency of at least 10 Hz to a target site of the vagus nerve via a transcutaneous approach.

20. The method of claim 18, wherein the target site of vagus nerve is a superficial neural target and the neuromodulation device comprises at least one intraneural energy delivery element.
